(19) United States
(12) Patent Application Publication
(10) Pub. No.: US 2026/0069370 A1
(43) Pub. Date: Mar. 12, 2026

(54) TECHNIQUES FOR IMPROVING PROCESSING OF VIDEO DATA USING MACHINE LEARNING MODELS

(71) Applicant: Verily Life Sciences LLC, Dallas, TX (US)

(72) Inventors: Daniel Hiranandani, Los Gatos, CA (US); Joëlle Barral, Mountain View, CA (US)

(21) Appl. No.: 19/391,881

(22) Filed: Nov. 17, 2025

Related U.S. Application Data
(63) Continuation of application No. 17/531,605, filed on Nov. 19, 2021.
(60) Provisional application No. 63/106,235, filed on Oct. 27, 2020.

(51) Int. Cl. A61B 34/30; G06F 13/38; G06N 20/00; G16H 10/60; G16H 30/20

(52) U.S. Cl. CPC A61B 34/30; G06F 13/382; G06N 20/00; G16H 10/60; G16H 30/20

(57) ABSTRACT

In some embodiments, a method of preparing video data for processing by a first machine learning model and a second machine learning model is provided. A computing device generates a first copy of the video data and a second copy of the video data. At least one of a frame rate, bit depth, first video resolution, or image encoding are different between the first copy and the second copy. The computing device processes the first copy of the video data using the first machine learning model to detect instances of a first item and processes the second copy of the video data using the second machine learning model to detect instances of a second item. A notification computing device is caused to provide at least one notification based on a detected instance of at least one of the first item or the second item.

### CROSS-REFERENCES TO RELATED APPLICATIONS

This application is a continuation of U.S. patent application Ser. No. 17/531,605, filed Nov. 19, 2021, and claims the benefit of Provisional Application No. 63/106,235, filed Oct. 27, 2020, the entire disclosures of which are hereby incorporated by reference herein for all purposes

### TECHNICAL FIELD

This disclosure relates generally to surgical technologies, and in particular but not exclusively, relates to using machine learning to analyze video data during a perioperative period.

### BACKGROUND

Robotic or computer assisted surgery uses robotic systems to aid in surgical procedures. Robotic surgery was developed as a way to overcome limitations (e.g., spatial constraints associated with a surgeon's hands, inherent shakiness of human movements, and inconsistency in human work product, etc.) of pre-existing surgical procedures. In recent years, the field has advanced greatly to limit the size of incisions, and reduce patient recovery time.

In the case of open surgery, autonomous instruments may replace traditional tools to perform surgical motions. Feedback-controlled motions may allow for smoother surgical steps than those performed by humans. For example, using a surgical robot for a step such as rib spreading may result in less damage to the patient's tissue than if the step were performed by a surgeon's hand. Additionally, surgical robots can reduce the amount of time in the operating room by requiring fewer steps to complete a procedure, and can make the required steps more efficient.

Even when guiding surgical robots, surgeons can easily be distracted by additional information provided to them during a surgical case. Any user interface (UI) that attempts to provide all relevant information to the surgeon at once may become crowded. Overlays have been shown to distract surgeons, causing inattention blindness, and actually hinder their surgical judgment rather than enhance it.

### BRIEF DESCRIPTION OF THE DRAWINGS

Non-limiting and non-exhaustive embodiments of the invention are described with reference to the following figures, wherein like reference numerals refer to like parts throughout the various views unless otherwise specified. Not all instances of an element are necessarily labeled so as not to clutter the drawings where appropriate. The drawings are not necessarily to scale, emphasis instead being placed upon illustrating the principles being described. To easily identify the discussion of any particular element or act, the most significant digit or digits in a reference number refer to the figure number in which that element is first introduced.

FIG. 1 illustrates a non-limiting example embodiment of a system for robot-assisted surgery, according to various aspects of the present disclosure.

FIG. 2 illustrates another non-limiting example embodiment of a system 200 for robot-assisted surgery according to various aspects of the present disclosure.

FIG. 3 is a block diagram that illustrates a non-limiting example embodiment of a machine learning (ML) processing computing device according to various aspects of the present disclosure.

FIG. 4 is a flowchart that illustrates a non-limiting example embodiment of a method of processing data to support a surgical procedure according to various aspects of the present disclosure.

### DETAILED DESCRIPTION

Surgeons often ask nurses for specific information that becomes important for them to know at specific times during a surgical case (e.g., medication the patient is under, available preoperative images). It takes time for nurses to find that information in computer systems, and it distracts the nurses from what they are doing. Sometimes the information cannot be found in a timely manner. Moreover, a main task of nurses is to predict which instrument the surgeon will need next and to have it ready when the surgeon asks for it. And sometimes the nurse may not accurately predict which instrument the surgeon needs.

In addition, surgical robots may be able to support apps, but these apps may not be easily discoverable, or surgeons may not want to interrupt what they are doing to open the right app at the right time, even if these apps might improve the surgery (similar to surgeons not using indocyanine green (ICG) to highlight critical structures because it takes time and effort).

Disclosed here is a system that recognizes which step the surgical procedure is at (temporally, spatially, or both), in real time, and provides cues to the surgeon based on the current, or an upcoming, surgical step. Surgical step recognition can be done in real time using machine learning. For example, machine learning may include using deep learning (applied frame by frame), or a combination of a convolutional neural net (CNN) and temporal sequence modeling (e.g., long short-term memory (LSTM)) for multiple spatial-temporal contexts of the current surgical step, which is then combined with the preceding classification result sequence, to enable real-time detection of the surgical step.

For example, the system can identify that the surgery is at “trocar placement” and provide a stadium view of the operation, or a schematic of where the next trocar should be placed, or provide guidance as to how a trocar should be inserted and/or which anatomical structures are expected under the skin and what the surgeon should be mindful of. Similarly, the system can identify that the surgery is about to begin tumor dissection and bring up the preoperative magnetic resonance image (MRI) or the relevant views from an anatomical atlas. In some embodiments, the system can estimate how long is left in the procedure. It can then provide an estimated “time of arrival” (when the procedure will be completed) as well as an “itinerary”, that is the list of steps left to complete the case. Having an estimate of the time left during the operation can help with operating room scheduling (e.g., when will staff rotate, when will the next case will start), family communication (e.g., when is surgery likely to be complete), and even with the case itself (e.g., the anesthesiologist starts waking the patient up about 30 min before the anticipated end of the case). Like with estimated time of arrival when driving a car, the estimated time left for the case can fluctuate over the course of the procedure. The system could also send automatic updates to other systems (e.g., the operating room scheduler).

Embodiments of the present disclosure provide functionality for recognizing anatomical structures within video data, recognizing surgical steps, predicting time remaining in an operation, and other functionality using a plurality of machine learning models. Typically, at least one machine learning model will be provided for each functionality provided by the system. The various machine learning models may also feed into each other, either directly having a first model's classification output used as input to another model, or indirectly by having a first model enhance features in the video data (e.g., by increasing brightness or contrast) and providing the enhanced video data to another model. What is needed are techniques for providing the proper data to each machine learning model in an efficient manner, such that low latency of the functionality can be maintained.

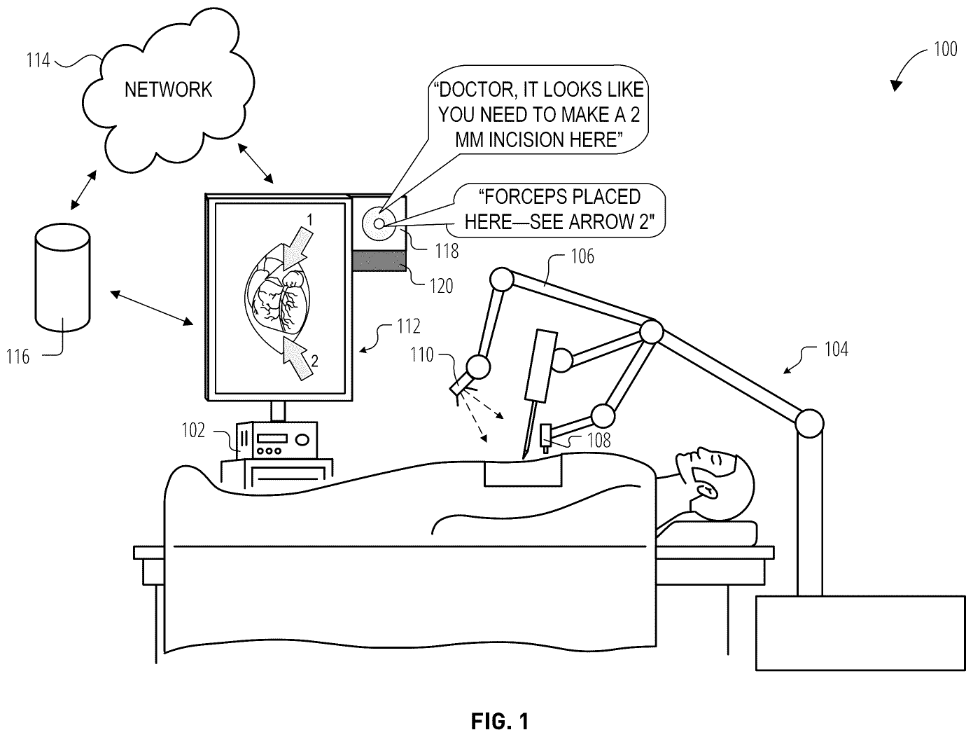

FIG. 1 illustrates a non-limiting example embodiment of a system for robot-assisted surgery, according to various aspects of the present disclosure. System 100 includes surgical robot 104 (including arms 106), camera 108, light source 110, display 112, controller 102, network 114, storage 116, loudspeaker 118, and microphone 120. All of these components may be coupled together to communicate either by wires or wirelessly.

As shown, surgical robot 104 may be used to hold surgical instruments (e.g., each arm 106 holds an instrument at the distal ends of arms 106) and perform surgery, diagnose disease, take biopsies, or conduct any other procedure a doctor could perform. Surgical instruments may include scalpels, forceps, cameras (e.g., camera 108, which may include a CMOS image sensor) or the like. While surgical robot 104 is illustrated as having three arms, one will appreciate that the illustrated surgical robot 104 is merely a cartoon illustration, and that a surgical robot 104 can take any number of shapes depending on the type of surgery needed to be performed and other requirements, including having more or fewer arms 106. Surgical robot 104 may be coupled to controller 102, network 114, and/or storage 116 either by wires or wirelessly. Furthermore, surgical robot 104 may be coupled (wirelessly or by wires) to a tactile user interface (UI) to receive instructions from a surgeon or doctor (e.g., the surgeon manipulates the UI to move and control the arms 106). The tactile user interface, and user of the tactile user interface, may be located very close to the surgical robot 104 and patient (e.g., in the same room) or may be located remotely, including but not limited to many miles apart. Thus, the surgical robot 104 may be used to perform surgery where a specialist is many miles away from the patient, and instructions from the surgeon are sent over the internet or secure network (e.g., network 114). Alternatively, the surgeon may be local and may simply prefer using surgical robot 104, for example because an embodiment of the surgical robot 104 may be able to better access a portion of the body than the hand of the surgeon.

As shown, an image sensor (in camera 108) is coupled to capture first images (e.g., a video stream or video data) of a surgical procedure, and display 112 is coupled to show second images (which may include a diagram of human anatomy, a preoperative image, or an annotated version of an image included in the first images). Controller 102 is coupled to camera 108 to receive the first images, and coupled to display 112 to output the second images. Controller 102 includes logic that when executed by controller 102 causes the system 100 to perform a variety of actions. For example, controller 102 may receive the first images from the image sensor, and identify a surgical step (e.g., initial incision, grasping tumor, cutting tumor away from surrounding tissue, close wound, etc.) in the surgical procedure from the first images. In some embodiments, identification can be not just from the videos alone, but also from other data coming from the surgical robot 104 (e.g., instruments, telemetry, logs, etc.), speech and/or other audio captured by microphone 120, and/or other types of data. The controller 102 may then display the second images on display 112 in response to identifying the surgical step.

In some embodiments, the second images may be used to guide the doctor during the surgery. For example, the system 100 may recognize that an initial incision for open heart surgery has been performed, and in response, display human anatomy of the heart for the relevant portion of the procedure. In some embodiments, the system 100 may recognize that the excision of a tumor is being performed, so the system 100 uses the display 112 to present a preoperative image (e.g., magnetic resonance image (MRI), X-ray, or computerized tomography (CT) scan, or the like) of the tumor to give the surgeon additional guidance. In some embodiments, the display 112 could show an image included in the first images that has been annotated. For example, after recognizing the surgical step, the system 100 may prompt the surgeon to complete the next step by showing the surgeon an annotated image. In the depicted embodiment, the system 100 annotated the image data output from the camera 108 by adding arrows to the images that indicate where the surgeon should place forceps, and where the surgeon should make an incision. Put another way, the image data may be altered to include an arrow or other highlighting that conveys information to the surgeon. In some embodiments, the image data may be altered to include a visual representation of how confident the system is that the system is providing the correct information (e.g., a confidence interval like “75% confidence”). For example, appropriate cutting might be at a specific position (a line) or within a region of interest.

In the depicted embodiment, microphone 120 is coupled to controller 102 to send voice commands from a user to controller 102. For example, the doctor could instruct the system 100 by saying “OK computer, display patient's pre-op MRI”. The system 100 would convert this spoken text into data, and recognize the command using natural language processing or the like. Similarly, loudspeaker 118 is coupled to the controller 102 to output audio. In the depicted example, the audio is prompting or cuing the surgeon to take a certain action “DOCTOR, IT LOOKS LIKE YOU NEED TO MAKE A 2 MM INCISION HERE”, and “FORCEPS PLACED HERE-SEE ARROW 2”. These audio commands may be output in response to the system 100 identifying the specific surgical step from the first images in the video data captured by the camera 108.

In the depicted embodiment, the logic may include one or more machine learning models trained to recognize surgical steps from the first images. The machine learning models may include at least one of a convolutional neural network (CNN) or temporal sequence model (e.g., long short-term memory (LSTM) model). The machine learning models may also, in some embodiments, utilize one or more of a deep learning algorithm, support vector machines (SVM), k-means clustering, or the like. The machine learning models may identify anatomical features by at least one of luminance, chrominance, shape, location in the body (e.g., relative to other organs, markers, etc.), or other features extracted from the video data. In some embodiments, the controller 102 may identify anatomical features in the video data using sliding window analysis. In some embodiments, the controller 102 stores at least some image frames from the first images in memory (e.g., local, on network 114, or in storage 116), to recursively train the machine learning algorithm. Thus, the system 100 brings a greater depth of knowledge and additional confidence to each new surgery.

It is also appreciated that the controller 102 may use one or more machine learning models to generate notifications relating to items identified by the machine learning models. For example, in some embodiments the controller 102 may annotate the image of the surgical procedure, included in the first images, by highlighting a piece of anatomy detected in the image (e.g., adding an arrow to the image, circling the anatomy with a box, changing the color of the anatomy, or the like). The machine learning model may also be used to highlight the location of a surgical step (e.g., where the next step of the procedure should be performed), highlight where a surgical instrument should be placed (e.g., where the scalpel should cut, where forceps should be placed next, etc.), or automatically optimize camera placement (e.g., move the camera 108 to a position that shows the most of the surgical area, or the like). The controller 102 may also use one or more machine learning models to estimate a remaining duration of the surgical procedure, in response to identifying the surgical step. For example, the controller 102 could determine that the final suturing step is about to occur, and recognize that, on average, there are 15 minutes until completion of the surgery. This may be used by the controller 102 to generate notifications that may update operating room calendars in real time, or inform family in the waiting room of the remaining time. Moreover, data about the exact length of a procedure could be collected and stored in memory, along with patient characteristics (e.g., body mass index, age, etc.) to better inform how long a surgery will take for subsequent surgeries of similar patients.

In the depicted embodiment, surgical robot 104 also includes light source 110 (e.g., LEDs or bulbs) to emit light and illuminate the surgical area. As shown, light source 110 is coupled to controller 102, and controller 102 may vary at least one of an intensity of the light emitted, a wavelength of the light emitted, or a duty ratio of the light source 110. In some embodiments, the light source 110 may emit visible light, IR light, UV light, or the like. Moreover, depending on the light emitted from light source 110, camera 108 may be able to discern specific anatomical features. For example, a contrast agent that binds to tumors and fluoresces under UV light may be injected into the patent. Camera 108 could record the fluorescent portion of the image, and controller 102 may identify that portion as a tumor.

In some embodiments, image/optical sensors (e.g., camera 108), pressure sensors (stress, strain, etc.) and the like are all used to control the surgical robot 104 and to ensure accurate motions and applications of pressure. Furthermore, these sensors may provide information to a processor (which may be included in surgical robot 104, controller 102, or another device) which uses a feedback loop to continually adjust the location, force, etc. applied by surgical robot 104. In some embodiments, sensors in the arms 106 of surgical robot 104 may be used to determine the position of the arms 106 relative to organs and other anatomical features. For example, surgical robot 104 may store and record coordinates of the instruments at the end of the arms 106, and these coordinates may be used in conjunction with video feed to determine the location of the arms 106 and anatomical features. It is appreciated that there are a number of different ways (e.g., from images, mechanically, time-of-flight laser systems, etc.) to calculate distances between components in the system 100 and any of these may be used to determine location, in accordance with the teachings of present disclosure.

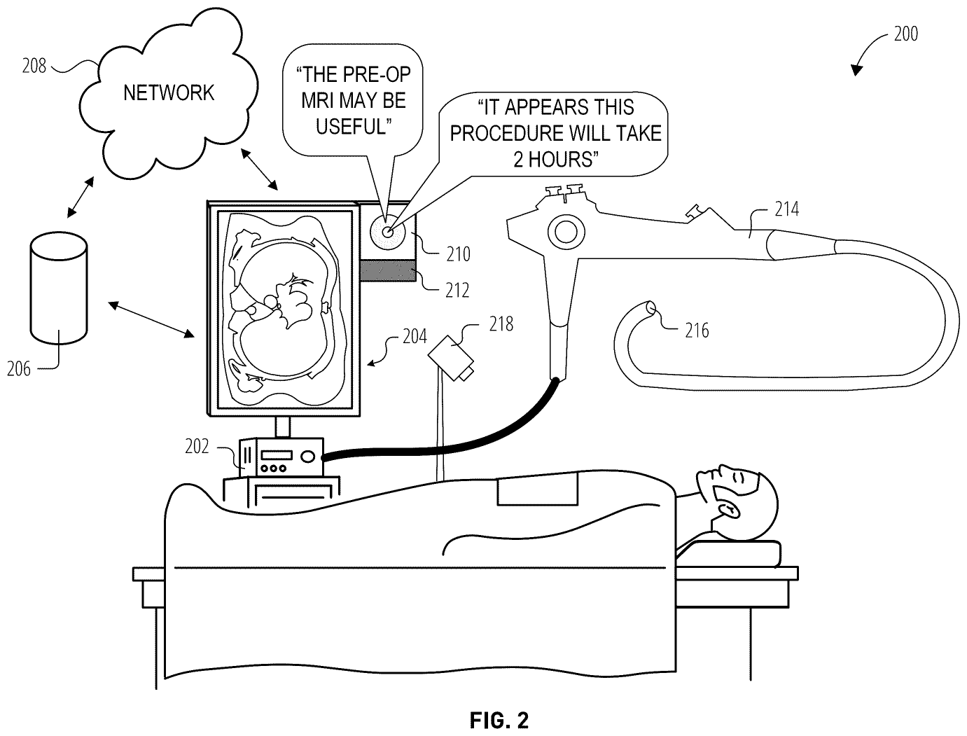

FIG. 2 illustrates another non-limiting example embodiment of a system 200 for robot-assisted surgery according to various aspects of the present disclosure. It is appreciated that system 200 includes many of the same features as system 100 of FIG. 1. Moreover, it is appreciated that the features illustrated in system 100 and system 200 are not mutually exclusive. For instance the endoscope in system 200 may be used in conjunction with, or may be part of, the surgical robot 104 in system 100. System 100 and system 200 have merely been drawn separately for ease of illustration.

In addition to the controller 202, display 204, storage 206, network 208, loudspeaker 210, and microphone 212 depicted in FIG. 1, FIG. 2 shows endoscope 214 (including a first camera 216, with an image sensor, disposed in the distal end of endoscope 214), and a second camera 218. In the depicted embodiment, endoscope 214 is coupled to controller 202. First images of the surgery may be provided by first camera 216 in endoscope 214, or by second camera 218, or both. It is appreciated that second camera 218 shows a higher-level view (viewing both the surgery and the operating room) of the surgical area than first camera 216 in endoscope 214.

In the depicted embodiment, the system 200 has identified (from the images captured by either first camera 216, second camera 218, or both first camera 216 and second camera 218) that the patients pre-op MRI may be useful for the surgery, and has subsequently brought up the MRI on display 204. System 200 also informed the doctor that it would do this by outputting the audio notification “THE PRE-OP MRI MAY BE USEFUL”. Similarly, after capturing first images of the surgery, the system 200 has recognized from the images that the surgery will take approximately two hours. The system 200 has presented a notification to the doctor of the ETA. In some embodiments, the system 200 may have automatically updated surgical scheduling software after determining the length of the procedure. The system 200 may also have announced the end time of the surgery to the waiting room or the lobby.

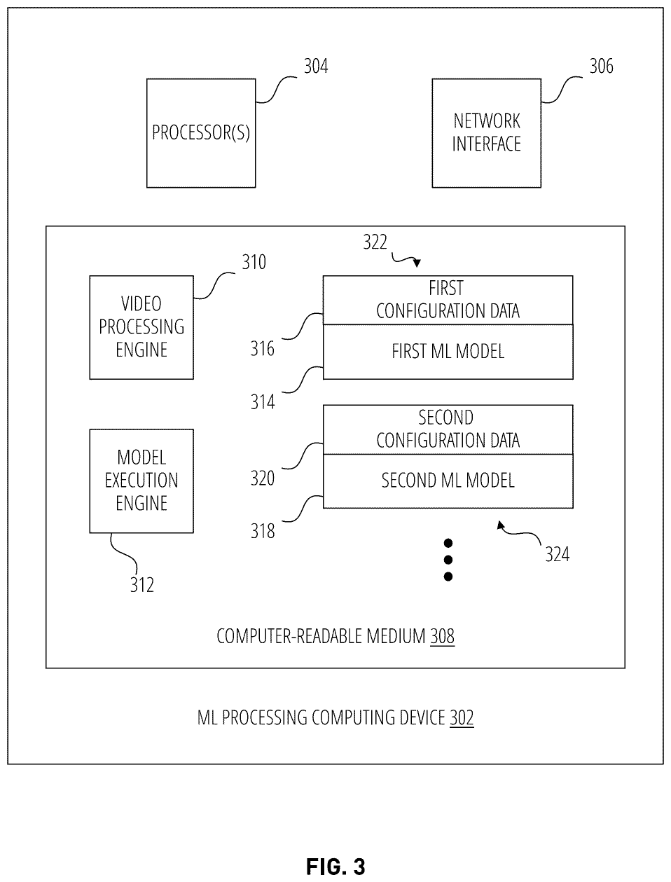

FIG. 3 is a block diagram that illustrates a non-limiting example embodiment of a machine learning (ML) processing computing device according to various aspects of the present disclosure. The ML processing computing device 302 is an example of a computing device that may be suitable for use as a controller 102 as illustrated in FIG. 1 or a controller 202 as illustrated in FIG. 2. The ML processing computing device 302 may be provided in any form factor, including but not limited to a desktop computing device, a laptop computing device, a rack-mount computing device, or a tablet computing device. In some embodiments, the ML processing computing device 302 may be incorporated into a controller of the surgical robot 104 or endoscope 214.

In some embodiments, the ML processing computing device 302 may be communicatively coupled to one or more cameras (including but not limited to the camera 108, the first camera 216, and/or the second camera 218) in order to receive video data. In some embodiments, the ML processing computing device 302 may be communicatively coupled to the cameras via a serial digital interface (SDI) connection, a high-definition multimedia interface (HDMI) connection, a USB connection, or any other suitable type of connection.

In some embodiments, instead of being directly coupled to the cameras, the ML processing computing device 302 may be communicatively coupled to a video capture computing device (not illustrated in FIG. 1 or FIG. 2) that is itself directly coupled to the cameras and generates video data based on signals received from the cameras. In some embodiments, the video capture computing device may receive raw signals directly from photodiodes of image sensors of the cameras, perform various image enhancement tasks on the raw signals (including but not limited to increasing a gain or applying one or more high-pass or low-pass filters), and provide either enhanced raw signals or video data generated based on the enhanced raw signals to the ML processing computing device 302. In some embodiments, the functionality of the ML processing computing device 302 and the video capture computing device may be combined into a single computing device. In some embodiments, the video capture computing device may include logic implemented in an application-specific integrated circuit (ASIC), a field-programmable gate array (FPGA), a graphics processing unit (GPU), or other hardware designed for fast processing of the signals and generation of video data.

As shown, the ML processing computing device 302 includes one or more processor(s) 304, a network interface 306, and a computer-readable medium 308. In some embodiments, the communicative coupling between the ML processing computing device 302 and the cameras (and/or between the ML processing computing device 302 and the optional video capture computing device, as well as between the optional video capture computing device and the cameras) may be via the network interface 306, which may use any suitable communication technology, including but not limited to wired technologies (including, but not limited to, USB, FireWire, Ethernet, SDI, HDMI, DVI, VGA, DisplayPort, and direct serial connections) and wireless technologies (including, but not limited to WiFi, WiMAX, and Bluetooth). In some embodiments, while a standard technology such as Ethernet may be used to transfer the video data between devices, care may be taken to transfer the video data in an optimal way. For example, in some embodiments, protocols such as HTTP or gRPC may be used to transfer the video data. As another example, lower-level protocols such as TCP or UDP packets may be used without higher-level protocols layered on top in order to improve efficiency. In some such embodiments, raw TCP sockets with additional length-based delimiting to denote where an image frame starts/ends may be used. As still another example, if two or more of the ML processing computing device 302, the video capture computing device, and the cameras are incorporated into a single device, the video data may be transferred using one or more inter-process communication techniques including but not limited to shared memory and/or Unix domain sockets.

As used herein, the terms “video signal” and “video data” refer to data that represents a sequence of images that, when presented, form a video stream. Though the systems disclosed herein are commonly described as processing video signals or video data, one will recognize that the processing described herein may also be applied to data in other formats, including but not limited to solitary images and groups of images that are provided separately instead of being combined in a video signal.

The illustrated computer-readable medium 308 may include one or more types of computer-readable media capable of storing logic executable by the processor(s) 304 and the illustrated machine learning models, including but not limited to one or more of a hard disk drive, a flash memory, an optical disc, an electrically erasable programmable read-only memory (EEPROM), random access memory (RAM), and read-only memory (ROM). In some embodiments, some portions of the logic may be provided by an application-specific integrated circuit (ASIC), a field-programmable gate array (FPGA), or other circuitry.

As illustrated, the computer-readable medium 308 stores logic for providing a video processing engine 310 and a model execution engine 312. As used herein, “engine” refers to logic embodied in hardware or software instructions, which can be written in one or more programming or scripting languages, including but not limited to C, C++, C#, COBOL, JAVA™, PHP, Perl, HTML, CSS, Javascript, VBScript, ASPX, Go, Python, shell scripting languages, and Rust. An engine may be compiled into executable programs or written in interpreted programming languages. Software engines may be callable from other engines or from themselves. Generally, the engines described herein refer to logical modules that can be merged with other engines, or can be divided into sub-engines. The engines can be implemented by logic stored in any type of computer-readable medium or computer storage device and be stored on and executed by one or more general purpose computers, thus creating a special purpose computer configured to provide the engine or the functionality thereof. The engines can be implemented by logic programmed into an application-specific integrated circuit (ASIC), a field-programmable gate array (FPGA), or another hardware device.

In some embodiments, the video processing engine 310 is configured to receive video data from the cameras (or from the video capture computing device) and to process it for submission to the machine learning models as described below. In some embodiments, the model execution engine 312 is configured to execute machine learning models stored by the computer-readable medium 308. As shown, the computer-readable medium 308 stores a first model container 322 and a second model container 324. In some embodiments, more than two model containers may be stored on the computer-readable medium 308. Typically, a separate model container is provided on the computer-readable medium 308 for each different item that may be detected from the image data by the ML processing computing device 302. As some non-limiting examples, a separate model container may be provided to identify a step in a medical procedure, to identify an anatomical structure, to identify a surgical tool, to identify proper and/or improper usage of a surgical tool during a medical procedure, to determine whether a surgical tool is inside or outside of a patient, or to estimate a time remaining in a surgical procedure.

Each model container includes configuration data and an ML model. As shown, the first model container 322 includes first configuration data 316 and a first ML model 314, while the second model container 324 includes second configuration data 320 and a second ML model 318. The configuration data indicates aspects of the data expected by the ML model included in the model container. For example, the configuration data may specify one or more of a frame rate, a bit depth, a video resolution, and an image frame encoding (e.g., PNG, JPG, BMP, or unencoded) for the video data to be processed by the ML model. As another example, the configuration data may also specify other data, including but not limited to telemetry data from the surgical robot 104 or endoscope 214 and/or patient-specific data from an electronic health record (EHR) system to be provided to the ML model.

In some embodiments, the ML model included in the model container (such as the first ML model 314 and the second ML model 318) provides information for executing a given machine learning model against the provided data. In some embodiments, the ML model may include architecture information (e.g., a number of layers and number of nodes per layer), parameter information (e.g., weights for edges between nodes), and/or other types of information that define a machine learning model provided to be executed by the model execution engine 312. In some embodiments, the ML model may also include the logic itself for executing the machine learning model processing, such that the model execution engine 312 can execute any type of ML model provided in a model container. In some embodiments, using model containers allows a given ML model to be distributed along with any particular dependencies used by the given ML model, including but not limited to specific versions of TensorFlow, CUDA, CUDNN, OpenCV, Python, or other dependencies. By using model containers that provide their own logic and configuration data, any type of machine learning models or combinations thereof may be used. Some non-limiting examples of types of machine learning models that may be used include convolutional neural networks (CNNs), support vector machines, k-means clustering models, deep learning models, and temporal sequence models (such as long short-term memory (LSTM) models). In some embodiments, the output of each model may indicate the presence or absence of an item, may indicate a location of an item within the video data, or may provide another type of notification regarding a presence or an absence of an item.

In some embodiments, a standard containerization platform may be used to provide and execute the model containers. For example, the model execution engine 312 may be (or may use) a Docker environment, and the model containers (including the first model container 322 and the second model container 324) may be provided in Docker containers.

Numerous technical benefits are provided by the use of the video processing engine 310, the model execution engine 312, and the model containers. For example, one goal of the system 100 and the system 200 is to provide timely information to support surgical procedures. In order to provide timely information, latency of the recognition of items by each machine learning models should be appropriate. For example, some notifications (like estimated time remaining notifications, or notifications related to surgical step identification) may be useful even if takes multiple seconds for the relevant machine learning models to process the video data, while other notifications (such as real-time annotations of anatomical structures on live video) may only be useful (that is, displayable without visible lag) if latency is on the order of milliseconds. By using the model containers that include configuration data, each model can be optimized to work on a minimum amount of video data in which the desired item can be detected, instead of each model having to process the full resolution, full bit depth, full frame rate video from the camera. Further, by downsampling the video data using the video processing engine 310 instead of another device, only one copy of the video data has to be transferred across the network to the ML processing computing device 302, thus avoiding inter-device communication bottlenecks.

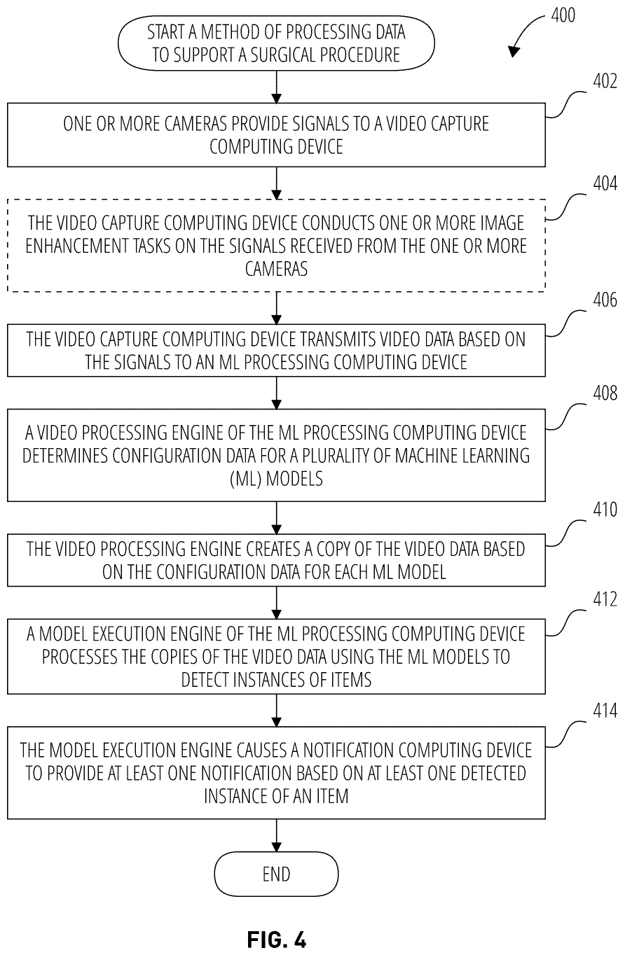

FIG. 4 is a flowchart that illustrates a non-limiting example embodiment of a method of processing data to support a surgical procedure according to various aspects of the present disclosure. The method 400 is an example of a technique that may be employed by the system 100, the system 200, or other similar systems in order to improve the processing of video data by various machine learning models.

From a start block, the method 400 proceeds to block 402, where one or more cameras, such as camera 108, first camera 216, or second camera 218, provide signals to a video capture computing device. In some embodiments, the signals are raw signals from an image sensor of the camera. In some embodiments, the signals are video data provided by the camera to the video capture computing device.

At optional block 404, the video capture computing device conducts one or more image enhancement tasks on the signals received from the one or more cameras. As described above, the video capture computing device may improve a gain, apply one or more band pass filters, or conduct other processing to improve the quality of the signals received from the one or more cameras. Optional block 404 is illustrated and described as optional because in some embodiments, the video capture computing device does not perform additional processing on the signals received from the one or more cameras, but instead generates video data directly from the signals received from the one or more cameras, or receives video data directly in the signals received from the one or more cameras.

At block 406, the video capture computing device transmits video data based on the signals to an ML processing computing device 302. In some embodiments, the video capture computing device may encode, compress, or otherwise process the video data in order to improve the transmission speed of the video data to the ML processing computing device 302.

At block 408, a video processing engine 310 of the ML processing computing device 302 determines configuration data for a plurality of machine learning (ML) models. In some embodiments, the video processing engine 310 may enumerate a plurality of model containers stored on the computer-readable medium 308 to determine configuration data for each of the model containers. For example, in the embodiment illustrated in FIG. 3, the video processing engine 310 may retrieve the first configuration data 316 from the first model container 322 and the second configuration data 320 from the second model container 324. Each configuration data may specify one or more aspects of input video data expected by its associated ML model, including but not limited to a video resolution, a bit depth, a frame rate, and an image encoding.

At block 410, the video processing engine 310 creates a copy of the video data based on the configuration data for each ML model. For example, if the first configuration data 316 specifies a first frame rate, a first video resolution, and a first bit depth, the video processing engine 310 will create a copy of the video data that has the specified first frame rate, video resolution, and bit depth. Typically, this will involve downsampling at least one of the frame rate, video resolution, and bit depth from the video data received by the ML processing computing device 302 to a lower value specified by the configuration data. The video processing engine 310 creates a separate copy for each different set of configuration data. For example, if the frame rate, video resolution, and bit depth for the first configuration data 316 and the second configuration data 320 all match, the video processing engine 310 would create only a single copy of the video data, but if any of these configuration settings were different, the video processing engine 310 would create separate copies of the video data.

In some embodiments, the creation of a copy causes a “true memory copy” to be created, in which an additional copy of the video data is created within memory. This additional copy is then provided to the model container for processing. In some embodiments, the creation of true memory copies may be minimized by storing the initial version of the video data to be stored in a shared memory, and the different formats desired by each model container are created as each model container accesses the shared memory.

At block 412, a model execution engine 312 of the ML processing computing device 302 processes the copies of the video data using the ML models to detect instances of items. In some embodiments, the model execution engine 312 may execute logic included in the model containers, using the appropriate copy of the video data as input, and receiving indications of identified items as output when the logic identifies such items. In some embodiments, the model execution engine 312 may provide other additional data to the ML models as appropriate, including but not limited to telemetry data from a surgical robot 104 or endoscope 214, and/or patient-specific data from an EHR system.

At block 414, the model execution engine 312 causes a notification computing device to provide at least one notification based on at least one detected instance of an item. Any suitable type of notification may be generated using any suitable kind of notification computing device. For example, if an anatomical structure is identified, then the notification may include an annotation on video data showing the identified location of the anatomical structure. This annotation may be displayed on, for example, the display 112, which is acting as or is coupled to a notification computing device. As another example, if an ML model determines an estimated time remaining in a procedure, the model execution engine 312 may update data within an electronic health record (EHR) or other system to indicate the estimated time the procedure will be completed. The EHR system (or other system), acting as a notification computing device, may then transmit alerts to other medical personnel, family members, or other appropriate recipients. As yet another example, if the ML model identifies a step in a procedure as occurring, the notification may include a preoperative image, an interoperative image, information from the EHR, or other information relevant to the step in the procedure. As still another example, a notification computing device may track an automated checklist indicating steps in the procedure, and/or pre- and post-procedure steps. As an ML model identifies steps being completed, the model execution engine 312 may cause the notification computing device to automatically complete items in the automated checklist.

The method 400 then proceeds to an end block and terminates. Though illustrated as terminating here for the sake of clarity, one will recognize that in many embodiments, the method 400 continues to run, with the cameras providing signals that are processed by the ML processing computing device 302 to identify items and generate notifications throughout the peri-operative period.

In the preceding description, numerous specific details are set forth to provide a thorough understanding of various embodiments of the present disclosure. One skilled in the relevant art will recognize, however, that the techniques described herein can be practiced without one or more of the specific details, or with other methods, components, materials, etc. In other instances, well-known structures, materials, or operations are not shown or described in detail to avoid obscuring certain aspects.

Reference throughout this specification to “one embodiment” or “an embodiment” means that a particular feature, structure, or characteristic described in connection with the embodiment is included in at least one embodiment of the present invention. Thus, the appearances of the phrases “in one embodiment” or “in an embodiment” in various places throughout this specification are not necessarily all referring to the same embodiment. Furthermore, the particular features, structures, or characteristics may be combined in any suitable manner in one or more embodiments.

The order in which some or all of the blocks appear in each method flowchart should not be deemed limiting. Rather, one of ordinary skill in the art having the benefit of the present disclosure will understand that actions associated with some of the blocks may be executed in a variety of orders not illustrated, or even in parallel.

The processes explained above are described in terms of computer software and hardware. The techniques described may constitute machine-executable instructions embodied within a tangible or non-transitory machine (e.g., computer) readable storage medium, that when executed by a machine will cause the machine to perform the operations described. Additionally, the processes may be embodied within hardware, such as an application specific integrated circuit (“ASIC”) or otherwise.

The above description of illustrated embodiments of the invention, including what is described in the Abstract, is not intended to be exhaustive or to limit the invention to the precise forms disclosed. While specific embodiments of, and examples for, the invention are described herein for illustrative purposes, various modifications are possible within the scope of the invention, as those skilled in the relevant art will recognize.

These modifications can be made to the invention in light of the above detailed description. The terms used in the following claims should not be construed to limit the invention to the specific embodiments disclosed in the specification. Rather, the scope of the invention is to be determined entirely by the following claims, which are to be construed in accordance with established doctrines of claim interpretation.

What is claimed is:

1. A computer-implemented method of preparing video data for processing by a first machine learning model and a second machine learning model, the actions comprising:
receiving, by a computing device, video data from a video capture computing device;
generating, by the computing device, a first copy of the video data and a second copy of the video data,
wherein the first copy of the video data has a first frame rate, a first bit depth, a first video resolution, and a first image encoding,
wherein the second copy of the video data has a second frame rate, a second bit depth, a second video resolution, and a second image encoding, and
wherein at least one of the first frame rate and second frame rate, the first bit depth and the second bit depth, the first video resolution and the second video resolution, or the first image encoding and the second image encoding are different from each other;
processing, by the computing device, the first copy of the video data using the first machine learning model to detect instances of a first item in the video data;
processing, by the computing device, the second copy of the video data using the second machine learning model to detect instances of a second item in the video data; and
causing, by the computing device, a notification computing device to provide at least one notification based on a detected instance of at least one of the first item or the second item.

2. The computer-implemented method of claim 1, wherein the first machine learning model is provided in a first container;
wherein the second machine learning model is provided in a second container;
wherein processing the first copy of the video data using the first machine learning model includes executing logic provided by the first container; and
wherein processing the second copy of the video data using the second machine learning model includes executing logic provided by the second container.

3. The computer-implemented method of claim 2, wherein the first frame rate, the first bit depth, the first video resolution, and the first image encoding are specified by configuration data associated with the first machine learning model; and
wherein the second frame rate, the second bit depth, the second video resolution, and the second image encoding are specified by configuration data associated with the second machine learning model.

4. The computer-implemented method of claim 3, wherein the configuration data associated with the first machine learning model is provided by the first container, and wherein the configuration data associated with the second machine learning model is provided by the second container.

5. The computer-implemented method of claim 1, wherein receiving the video data from the video capture computing device includes receiving the video data via a serial digital interface (SDI) connection, a high-definition multimedia interface (HDMI) connection, or a USB connection.

6. The computer-implemented method of claim 1, wherein the first item includes a presence of a surgical instrument, an occurrence of a surgical step, an anatomical structure, a determination of whether a surgical instrument is inside or outside of a patient, or an estimation of time remaining in a surgical procedure.

7. The computer-implemented method of claim 1, wherein the at least one notification includes a diagram of human anatomy, a preoperative image, an intraoperative image, an annotated intraoperative image, an identification of a surgical step, a display of estimated time remaining, a change to a checklist item, or a data update in an electronic health record (EHR).

8. A non-transitory computer-readable medium having computer-executable instructions stored thereon that, in response to execution by one or more processors of a computing device, cause the computing device to perform actions for preparing video data for processing by a first machine learning model and a second machine learning model, the actions comprising:
receiving, by the computing device, video data from a video capture computing device;
generating, by the computing device, a first copy of the video data and a second copy of the video data,
wherein the first copy of the video data has a first frame rate, a first bit depth, a first video resolution, and a first image encoding,
wherein the second copy of the video data has a second frame rate, a second bit depth, a second video resolution, and a second image encoding, and
wherein at least one of the first frame rate and second frame rate, the first bit depth and the second bit depth, the first video resolution and the second video resolution, or the first image encoding and the second image encoding are different from each other;
processing, by the computing device, the first copy of the video data using a first machine learning model to detect instances of a first item in the video data;
processing, by the computing device, the second copy of the video data using a second machine learning model to detect instances of a second item in the video data; and
causing, by the computing device, a notification computing device to provide at least one notification based on a detected instance of at least one of the first item or the second item.

9. The non-transitory computer-readable medium of claim 8, wherein the first machine learning model is provided in a first container;
wherein the second machine learning model is provided in a second container;
wherein processing the first copy of the video data using the first machine learning model includes executing logic provided by the first container; and
wherein processing the second copy of the video data using the second machine learning model includes executing logic provided by the second container.

10. The non-transitory computer-readable medium of claim 9, wherein the first frame rate, the first bit depth, the first video resolution, and the first image encoding are specified by configuration data associated with the first machine learning model; and
wherein the second frame rate, the second bit depth, the second video resolution, and the second image encoding are specified by configuration data associated with the second machine learning model.

11. The non-transitory computer-readable medium of claim 10, wherein the configuration data associated with the first machine learning model is provided by the first container, and wherein the configuration data associated with the second machine learning model is provided by the second container.

12. The non-transitory computer-readable medium of claim 8, wherein receiving the video data from the video capture computing device includes receiving the video data via a serial digital interface (SDI) connection, a high-definition multimedia interface (HDMI) connection, or a USB connection.

13. The non-transitory computer-readable medium of claim 8, wherein the first item includes a presence of a surgical instrument, an occurrence of a surgical step, an anatomical structure, a determination of whether a surgical instrument is inside or outside of a patient, or an estimation of time remaining in a surgical procedure.

14. The non-transitory computer-readable medium of claim 8, wherein the at least one notification includes a diagram of human anatomy, a preoperative image, an intraoperative image, an annotated intraoperative image, an identification of a surgical step, a display of estimated time remaining, a change to a checklist item, or a data update in an electronic health record (EHR).

15. A system, comprising:
an image sensor;
a video capture computing device configured to receive signals from the image sensor and to generate video data;
a notification computing device; and
a machine learning (ML) processing computing device communicatively coupled to the video capture computing device and the notification computing device;
wherein the ML processing computing device includes logic that, in response to execution by the ML processing computing device, causes the system to perform actions including:
receiving, by the computing device, video data from a video capture computing device;
generating, by the computing device, a first copy of the video data and a second copy of the video data,
wherein the first copy of the video data has a first frame rate, a first bit depth, a first video resolution, and a first image encoding,
wherein the second copy of the video data has a second frame rate, a second bit depth, a second video resolution, and a second image encoding, and
wherein at least one of the first frame rate and second frame rate, the first bit depth and the second bit depth, the first video resolution and the second video resolution, or the first image encoding and the second image encoding are different from each other;
processing, by the computing device, the first copy of the video data using a first machine learning model to detect instances of a first item in the video data;
processing, by the computing device, the second copy of the video data using a second machine learning model to detect instances of a second item in the video data; and
causing, by the computing device, a notification computing device to provide at least one notification based on a detected instance of at least one of the first item or the second item.

16. The system of claim 15, wherein the first machine learning model is provided in a first container;
wherein the second machine learning model is provided in a second container;
wherein processing the first copy of the video data using the first machine learning model includes executing logic provided by the first container; and
wherein processing the second copy of the video data using the second machine learning model includes executing logic provided by the second container.

17. The system of claim 16, wherein the first frame rate, the first bit depth, the first video resolution, and the first image encoding are specified by configuration data associated with the first machine learning model;
wherein the second frame rate, the second bit depth, the second video resolution, and the second image encoding are specified by configuration data associated with the second machine learning model;
wherein the configuration data associated with the first machine learning model is provided by the first container; and
wherein the configuration data associated with the second machine learning model is provided by the second container.

18. The system of claim 15, wherein receiving the video data from the video capture computing device includes receiving the video data via a serial digital interface (SDI) connection, a high-definition multimedia interface (HDMI) connection, or a USB connection.

19. The system of claim 15, wherein the first item includes a presence of a surgical instrument, an occurrence of a surgical step, an anatomical structure, a determination of whether a surgical instrument is inside or outside of a patient, or an estimation of time remaining in a surgical procedure.

20. The system of claim 15, wherein the at least one notification includes a diagram of human anatomy, a preoperative image, an intraoperative image, an annotated intraoperative image, an identification of a surgical step, a display of estimated time remaining, a change to a checklist item, or a data update in an electronic health record (EHR).
